(19) United States
(12) Patent Application Publication
(10) Pub. No.: US 2006/0246009 A1
(43) Pub. Date: Nov. 2, 2006

(54) Novel Crystalline Forms of Tiotropium Bromide

(75) Inventors: Sherry Morissette, Lexington, MA (US); Mark Tawa, Lexington, MA (US); Mark Oliveira, Lexington, MA (US)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 11/381,079

(22) Filed: May 1, 2006

Related U.S. Application Data
(60) Provisional application No. 60/676,760, filed on May 2, 2005.

(51) Int. Cl. A61K 31/4745; C07D 491/08; A61K 9/14

(52) U.S. Cl. 424/46; 514/291; 546/91

(57) ABSTRACT

The invention relates to new crystalline forms of tiotropium bromide, processes for preparing them and their use for preparing a pharmaceutical composition for the treatment of respiratory complaints, particularly for the treatment of COPD (chronic obstructive pulmonary disease) and asthma.

### RELATED APPLICATIONS

This application claims benefit and priority to U.S. provisional application No. 60/676,760, filed May 2, 2005, the content of which is hereby incorporated by reference in its entirety.

### FIELD OF THE INVENTION

The invention relates to new crystalline forms of tiotropium bromide, processes for preparing them and their use for preparing a pharmaceutical composition for the treatment of respiratory complaints, particularly for the treatment of COPD (chronic obstructive pulmonary disease) and asthma.

### BACKGROUND TO THE INVENTION

Tiotropium bromide is known from European Patent Application EP 418 716 A1 and has the following chemical structure:

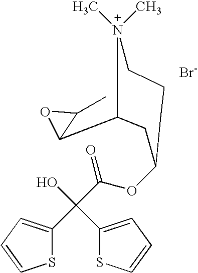

Tiotropium bromide is a highly effective anticholinergic with a long-lasting effect, which may be used to treat respiratory complaints, particularly COPD (chronic obstructive pulmonary disease) and asthma. By tiotropium is meant the free ammonium cation.

Tiotropium bromide is preferably administered by inhalation. Suitable inhalable powders packed into appropriate capsules (inhalettes) may be used. Alternatively, it may be administered by the use of suitable inhalable aerosols. These also include powdered inhalable aerosols which contain, for example, HFA134a, HFA227 or mixtures thereof as propellent gas.

The correct manufacture of the abovementioned compositions which are suitable for use for the administration of a pharmaceutically active substance by inhalation is based on various parameters which are connected with the nature of the active substance itself. In pharmaceutical compositions which are used like tiotropium bromide in the form of inhalable powders or inhalable aerosols, the crystalline active substance is used in ground (micronised) form for preparing the formulation. Since the pharmaceutical quality of a pharmaceutical formulation requires that the active substance should always have the same crystalline modification, the stability and properties of the crystalline active substance are subject to stringent requirements from this point of view as well. It is particularly desirable that the active substance should be prepared in the form of a uniform and clearly defined crystalline modification. It is also particularly desirable that the active substance be prepared in a crystalline form which is characterised by a high degree of stability even over long storage periods. The lower the tendency of a crystalline modification to absorb moisture, for example, the greater the physical stability of its crystal structure.

The aim of the invention is therefore to provide new stable crystal forms of the compound tiotropium bromide which meet the high demands mentioned above that are made of any pharmaceutically active substance.

### BRIEF DESCRIPTION OF THE DRAWINGS

FIG. 1: FIG. 1: X-ray powder diffraction of anhydrous crystalline tiotropium bromide

FIG. 2: Differential Scanning Calorimetry diagram of crystalline tiotropium bromide anhydrate

FIG. 3: X-ray powder diffraction of crystalline methanol solvate of tiotropium bromide

FIG. 4: DSC diagram of crystalline methanol solvate of tiotropium bromide

FIG. 5: X-ray powder diffraction of crystalline ethanol solvate of tiotropium bromide

FIG. 6: DSC diagram of crystalline ethanol solvate of tiotropium bromide

FIG. 7: X-ray powder diffraction of crystalline isopropanol solvate of tiotropium bromide

FIG. 8: DSC diagram of crystalline isopropanol solvate of tiotropium bromide

FIG. 9: X-ray powder diffraction of crystalline THF solvate of tiotropium bromide

FIG. 10: DSC diagram of crystalline THF solvate of tiotropium bromide

FIG. 11: X-ray powder diffraction of crystalline 1,4-dioxane solvate of tiotropium bromide

FIG. 12: DSC diagram of crystalline 1,4-dioxane solvate of tiotropium bromide

FIG. 13: X-ray powder diffraction of crystalline DMF solvate of tiotropium bromide

FIG. 14: X-ray powder diffraction of crystalline methylene chloride/methyl ethyl ketone solvate of tiotropium bromide

FIG. 15: DSC diagram of crystalline methylene chloride/methyl ethyl ketone solvate of tiotropium bromide

FIG. 16: X-ray powder diffraction of crystalline 1-butanol solvate of tiotropium bromide

FIG. 17: Exploded view of a preferred inhaler for administration of the pharmaceutical compositions described herein

### DETAILED DESCRIPTION OF THE INVENTION

It has been found that, depending on the choice of the conditions which may be used during the purification of the crude product obtained after industrial production, tiotropium bromide may be obtained in different crystalline modifications.

It has been found that these different modifications can be decisively obtained by the choice of solvents used for the crystallisation and by the choice of the operating conditions selected during the crystallisation process.

It has surprisingly been found that, starting from the monohydrate of tiotropium bromide, which can be obtained in crystalline form by choosing specific reaction conditions and which was described in the prior art for the first time in WO 02/30928, several crystal modifications of tiotropium bromide may be obtained which meet the high requirements set out above and thereby solve the problem underlying the present invention.

Accordingly, the present invention relates to a novel crystalline anhydrous tiotropium bromide. Any reference made within the scope of the present invention to the term tiotropium bromide anhydrate is to be regarded as a reference to the novel crystalline anhydrous tiotropium bromide according to the invention.

In another aspect the present invention relates to a method of preparing the new crystalline form of anhydrous tiotropium bromide which is explained by way of example in the experimental section that follows.

The crystalline tiotropium bromide anhydrate according to the invention is characterised in that in the X-ray powder diagram it has the following characteristic peaks (most dominant ones) with the values d=9.84 Å; 8.89 Å; 8.10 Å; 7.54 Å; 5.89 Å; 4.90 Å; 4.84 Å, and 4.05 Å. For more details see table 1.

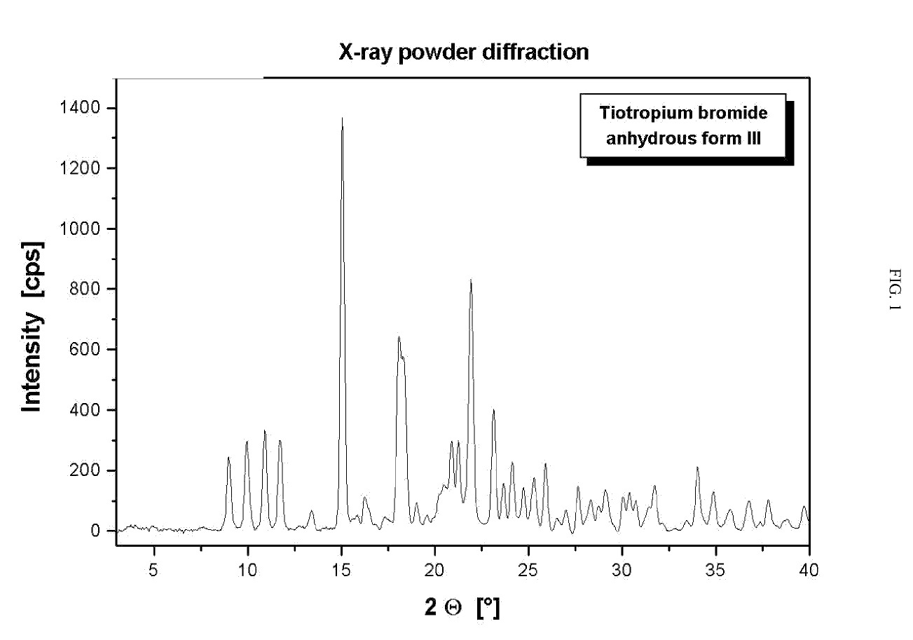

The X-ray powder diagram of the crystalline tiotropium bromide anhydrate according to the invention is depicted in FIG. 1.

Furthermore, the crystalline tiotropium bromide anhydrate according to the invention is characterised by an endothermic peak at 230° C. occurring during thermal analysis using DSC, indicating melting of this form.

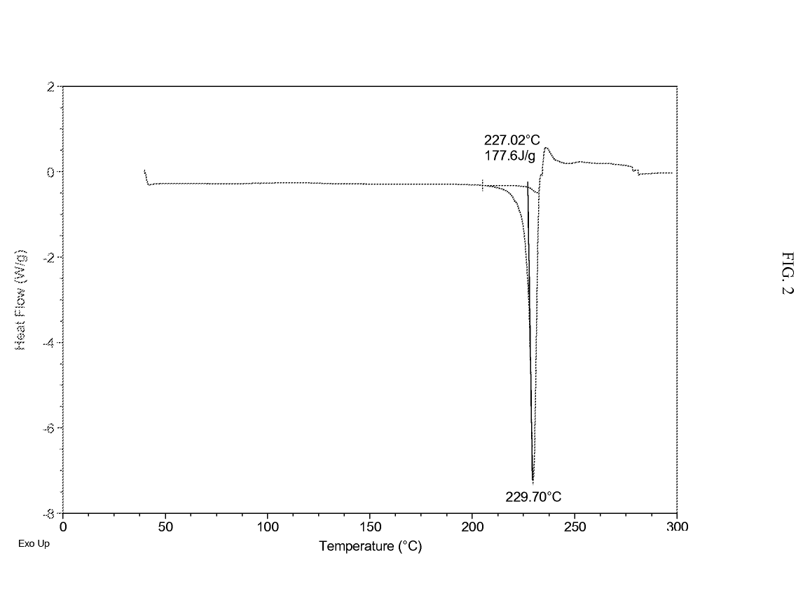

The DSC diagram of the crystalline tiotropium bromide anhydrate according to the invention is depicted in FIG. 2.

In another embodiment, the present invention relates to novel crystalline solvates of tiotropium bromide. One aspect of the invention is directed to a crystalline methanol solvate of tiotropium bromide. In another aspect the present invention relates to a method of preparing the new crystalline methanol solvate of tiotropium bromide which is explained by way of example in the experimental section that follows.

The crystalline methanol solvate of tiotropium bromide according to the invention is characterised in that in the X-ray powder diagram it has the following characteristic peaks (most dominant ones) with the values d=9.00 Å; 8.10 Å; 6.58 Å; 5.77 Å; 4.94 Å; 4.50 Å; 4.24 Å, and 4.14 Å. For more details see table 2.

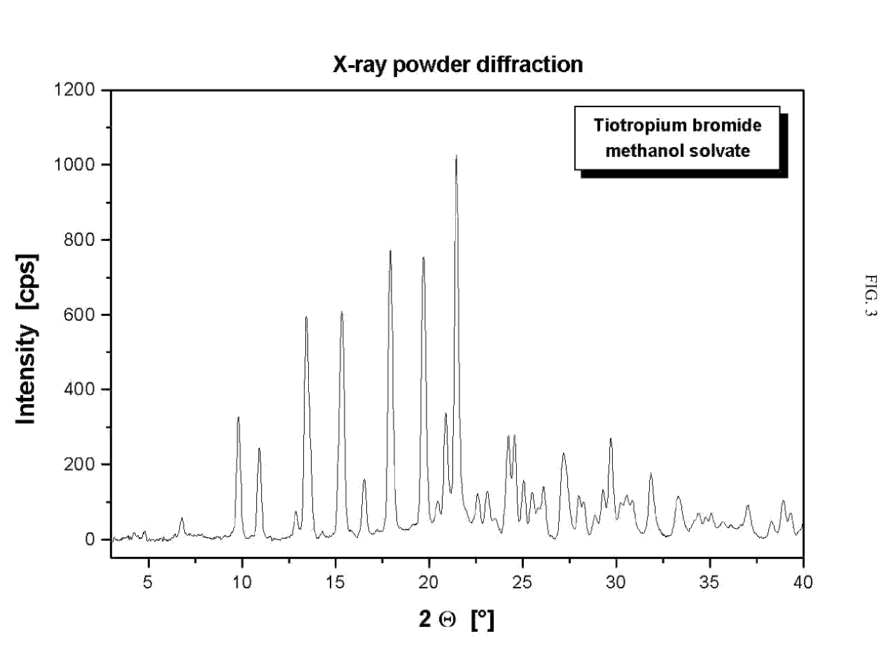

The X-ray powder diagram of the crystalline methanol solvate of tiotropium bromide is depicted in FIG. 3.

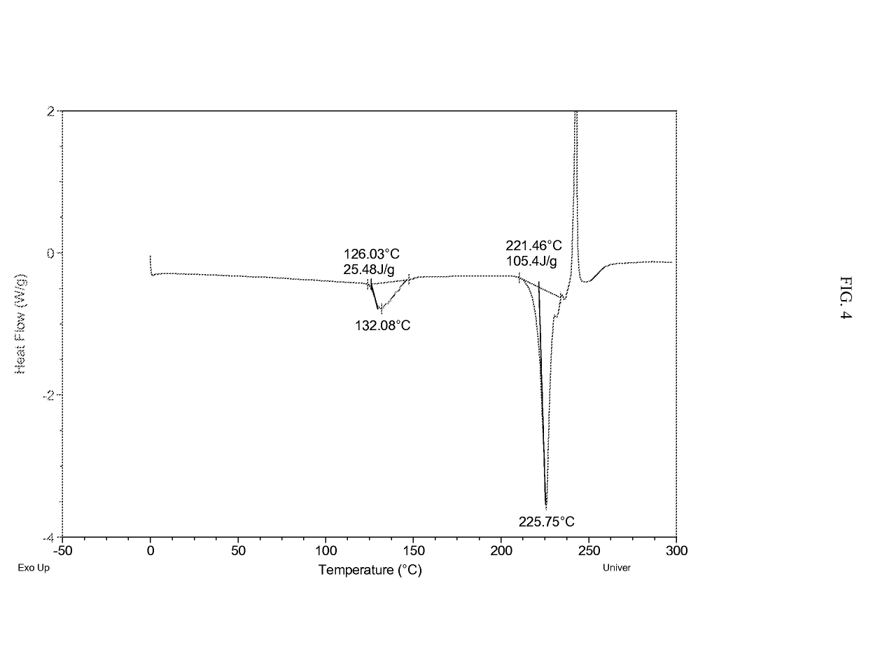

Furthermore, the crystalline methanol solvate of tiotropium bromide according to the invention is characterised by a strong endothermic peak at 226° C. occurring during thermal analysis using DSC, indicating melting of this form. An additional small endothermic event appears at 132° C. at which desolvation is observed. The DSC diagram of the crystalline methanol solvate of tiotropium bromide according to the invention is depicted in FIG. 4.

In a yet another embodiment, the present invention relates to a novel crystalline ethanol solvate of tiotropium bromide. In another aspect the present invention relates to a method of preparing the new crystalline ethanol solvate of tiotropium bromide which is explained by way of example in the experimental section that follows.

The crystalline ethanol solvate of tiotropium bromide according to the invention is characterised in that in the X-ray powder diagram it has the following characteristic peaks (most dominant ones) with the values d=8.91 Å; 8.01 Å; 6.60 Å; 5.78 Å; 4.90 Å; 4.46 Å; 4.24 Å, and 4.15 Å. For more details see table 3.

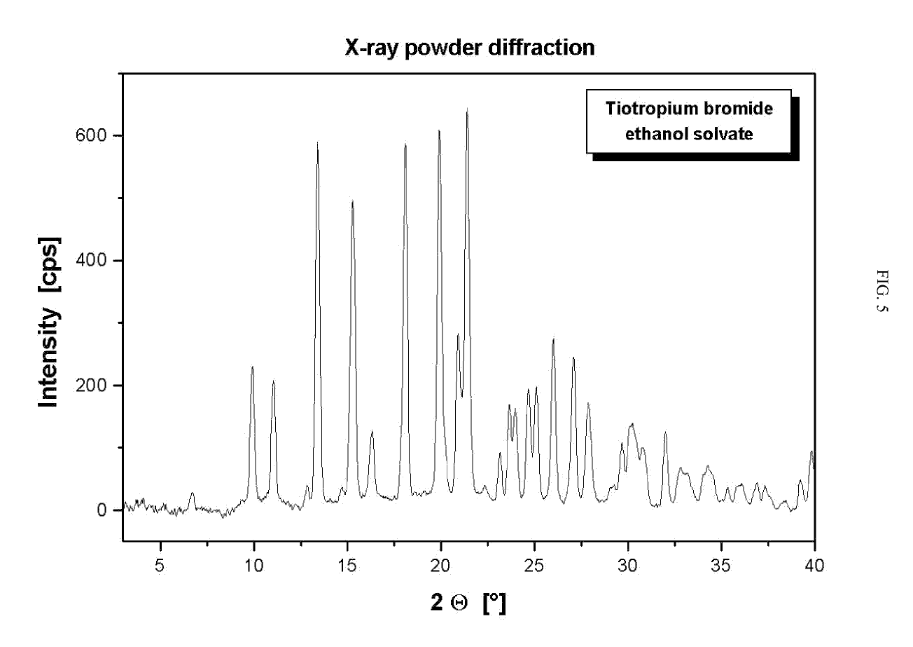

The X-ray powder diagram of the crystalline ethanol solvate of tiotropium bromide is depicted in FIG. 5.

Furthermore, the crystalline ethanol solvate of tiotropium bromide according to the invention is characterised by an endothermic peak at 226° C. occurring during thermal analysis using DSC, indicating melting of this form. An additional small endothermic event appears at 157° C. at which desolvation is observed.

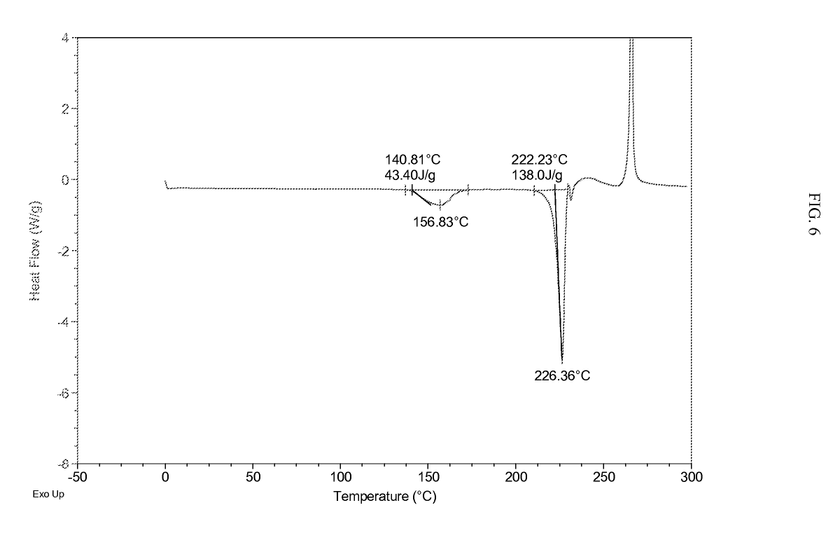

The DSC diagram of the crystalline ethanol solvate of tiotropium bromide according to the invention is depicted in FIG. 6.

In a yet another embodiment, the present invention relates to a novel crystalline isopropanol solvate of tiotropium bromide. In another aspect the present invention relates to a method of preparing the new crystalline isopropanol solvate of tiotropium bromide which is explained by way of example in the experimental section that follows.

The crystalline isopropanol solvate of tiotropium bromide according to the invention is characterised in that in the X-ray powder diagram it has the following characteristic peaks (most dominant ones) with the values d=8.96 Å; 8.06 Å; 6.66 Å; 5.80 Å; 4.91 Å; 4.48 Å; 4.28 Å, and 4.17 Å. For more details see table 4.

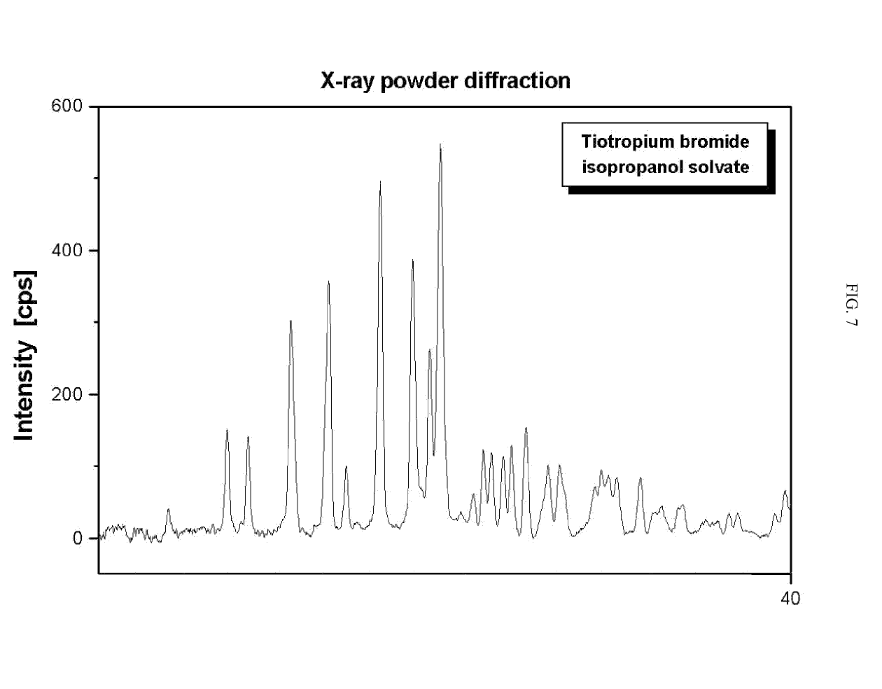

The X-ray powder diagram of the crystalline isopropanol solvate of tiotropium bromide is depicted in FIG. 7.

Furthermore, the crystalline isopropanol solvate of tiotropium bromide according to the invention is characterised by an exothermic peak at 264° C. occurring during thermal analysis using DSC, indicating thermal decomposition of this form. Two additional smaller endothermic events appear at 117° C. and 214° C. at which desolvation and melting is observed.

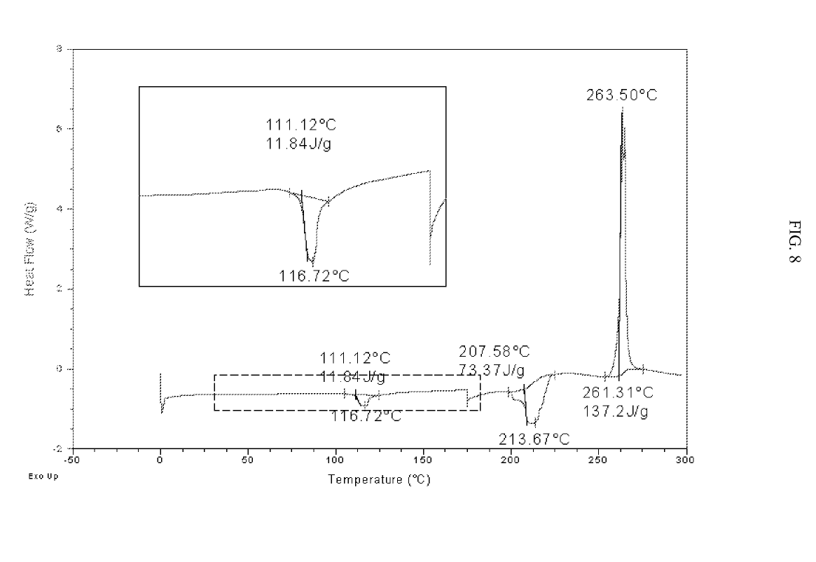

The DSC diagram of the crystalline isopropanol solvate of tiotropium bromide according to the invention is depicted in FIG. 8.

In a yet another embodiment, the present invention relates to a novel crystalline THF (tetrahydrofuran) solvate of tiotropium bromide. In another aspect the present invention relates to a method of preparing the new crystalline THF solvate of tiotropium bromide which is explained by way of example in the experimental section that follows.

The crystalline THF solvate of tiotropium bromide according to the invention is characterised in that in the X-ray powder diagram it has the following characteristic peaks (most dominant ones) with the values d=8.97 Å; 8.03 Å; 6.60 Å; 5.80 Å; 4.92 Å; 4.48 Å; 4.30 Å, and 4.15 Å. For more details see table 5.

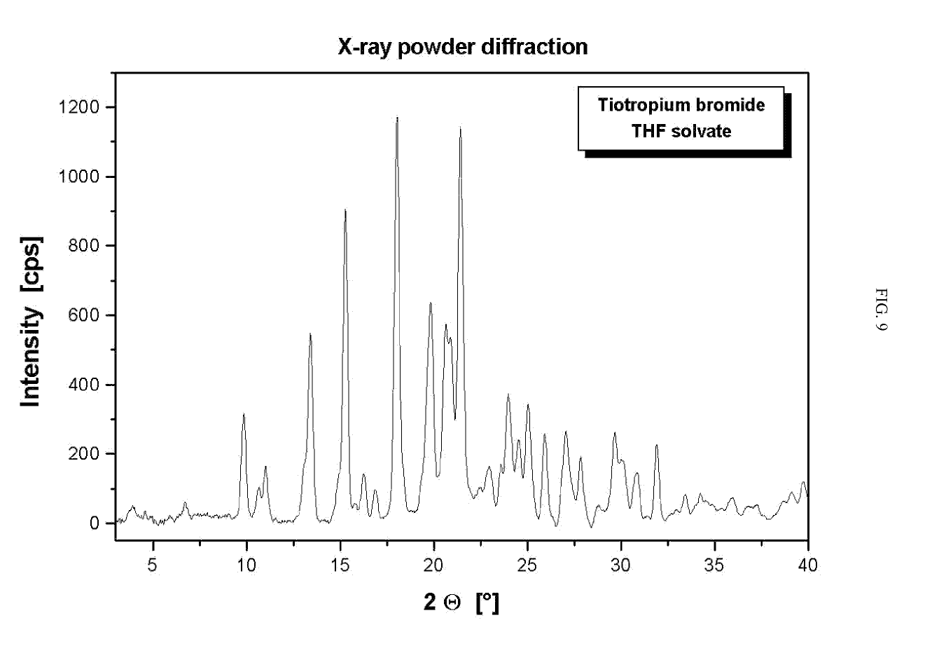

The X-ray powder diagram of the crystalline THF solvate of tiotropium bromide is depicted in FIG. 9.

Furthermore, the crystalline THF solvate of tiotropium bromide according to the invention is characterised by an endothermic peak at 216° C., indicating melting of the form, and an exothermic peak at 275° C., indicating thermal decomposition, occurring during thermal analysis using DSC An additional small endothermic event appears at 125° C. at which desolvation is observed.

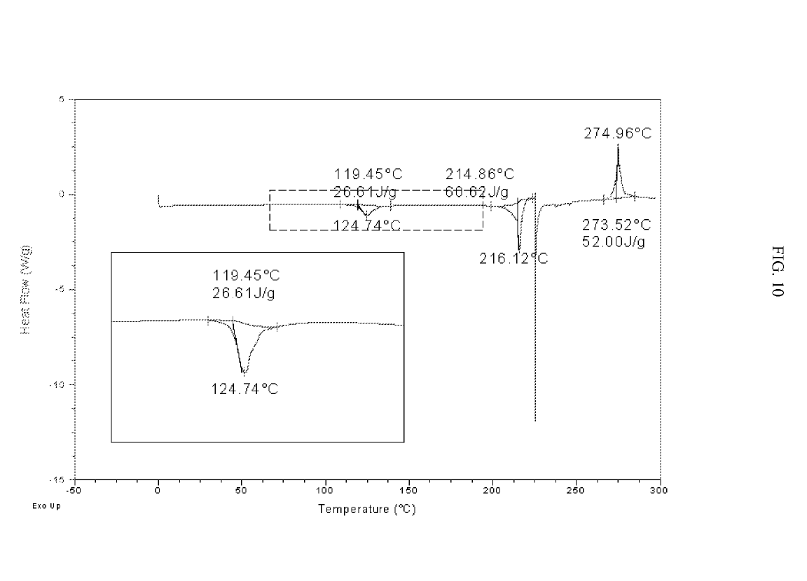

The DSC diagram of the crystalline THF solvate of tiotropium bromide according to the invention is depicted in FIG. 10.

In a yet another embodiment, the present invention relates to a novel crystalline 1,4-dioxane solvate of tiotropium bromide. In another aspect the present invention relates to a method of preparing the new crystalline 1,4-dioxane solvate of tiotropium bromide which is explained by way of example in the experimental section that follows.

The crystalline 1,4-dioxane solvate of tiotropium bromide according to the invention is characterised in that in the X-ray powder diagram it has the following characteristic peaks (most dominant ones) with the values d=8.92 Å; 8.08 Å; 6.59 Å; 5.79 Å; 4.92 Å; 4.51 Å; 4.27 Å, and 4.15 Å. For more details see table 6.

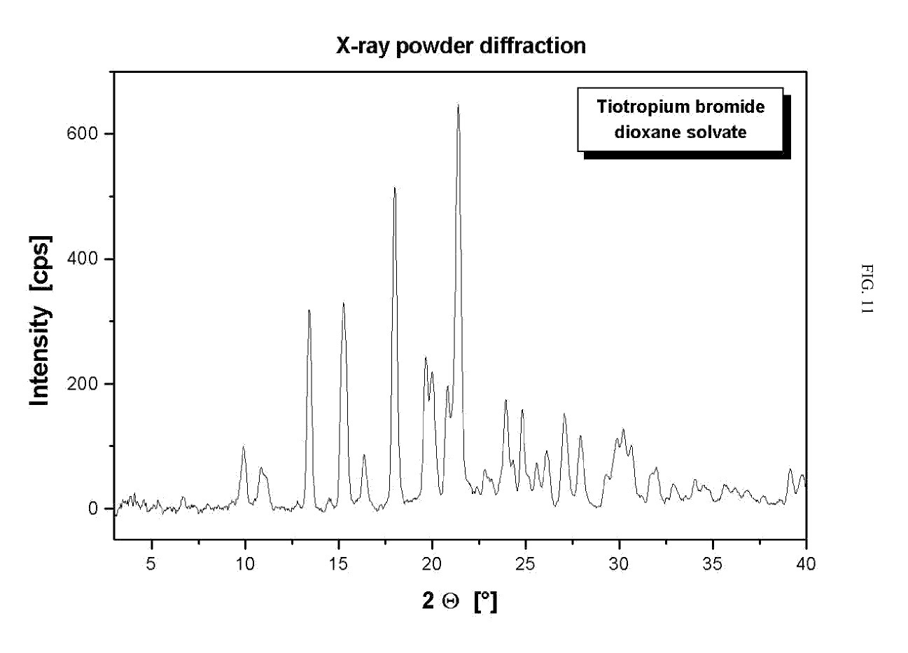

The X-ray powder diagram of the crystalline 1,4-dioxane solvate of tiotropium bromide is depicted in FIG. 11.

Furthermore, the crystalline 1,4-dioxane solvate of tiotropium bromide according to the invention is characterised by an endothermic peak at 223° C. occurring during thermal analysis using DSC, indicating melting of this form. An additional small endothermic event appears at 191° C. at which desolvation is observed

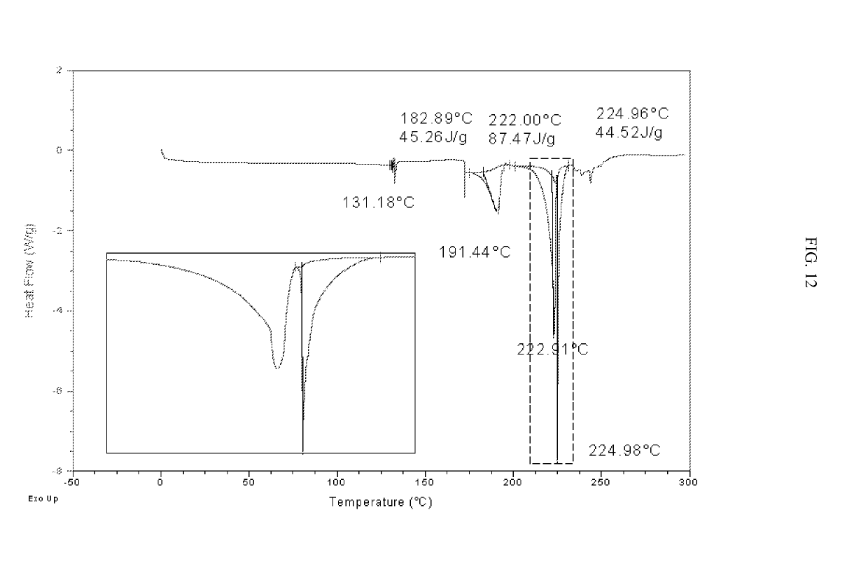

The DSC diagram of the crystalline 1,4-dioxane solvate of tiotropium bromide according to the invention is depicted in FIG. 12.

In a yet another embodiment, the present invention relates to a novel crystalline dimethylformamide (DMF) solvate of tiotropium bromide. In another aspect the present invention relates to a method of preparing the new crystalline DMF solvate of tiotropium bromide which is explained by way of example in the experimental section that follows.

The crystalline DMF solvate of tiotropium bromide according to the invention is characterised in that in the X-ray powder diagram it has the following characteristic peaks (most dominant ones) with the values d=10.03 Å, 8.95 Å; 8.02 Å; 7.54 Å, 6.82 Å, 6.55 Å; 5.78 Å; 5.69 Å, 5.00 Å, 4.94 Å; 4.48 Å; 4.21 Å, and 4.11 Å. For more details see table 7.

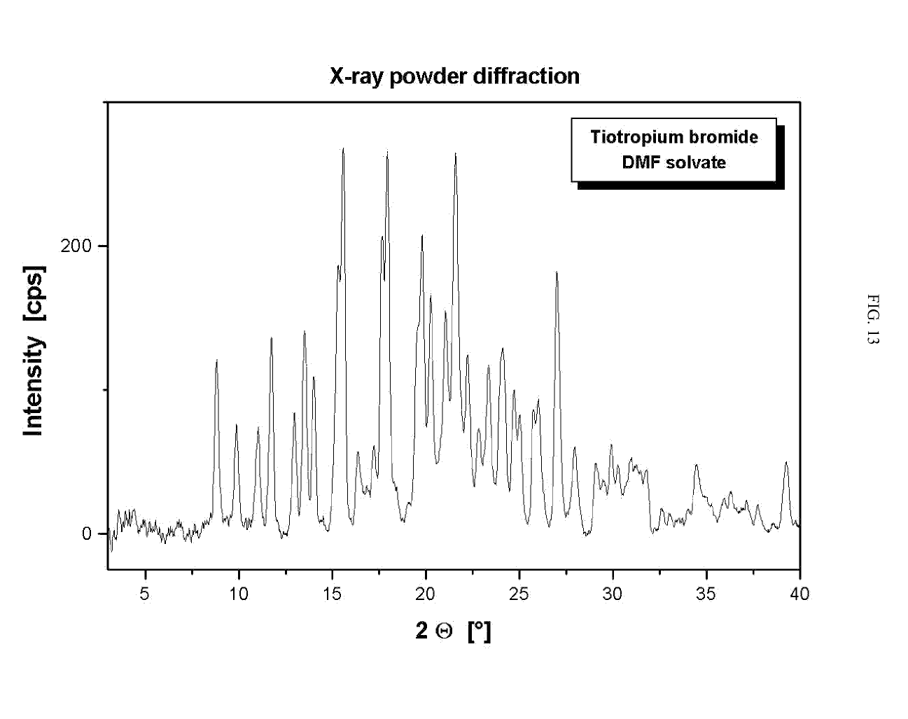

The X-ray powder diagram of the crystalline DMF solvate of tiotropium bromide is depicted in FIG. 13.

In a yet another embodiment, the present invention relates to a novel crystalline mixed methylene chloride/methyl ethyl ketone solvate of tiotropium bromide. In another aspect the present invention relates to a method of preparing the new crystalline mixed methylene chloride/methyl ethyl ketone of tiotropium bromide which is explained by way of example in the experimental section that follows.

The crystalline mixed methylene chloride/methyl ethyl ketone solvate of tiotropium bromide according to the invention is characterised in that in the X-ray powder diagram it has the following characteristic peaks (most dominant ones) with the values d=8.91 Å; 8.02 Å; 6.56 Å; 5.79 Å; 5.43 Å, 4.91 Å; 4.45 Å; 4.22 Å, and 4.13 Å. For more details see table 8.

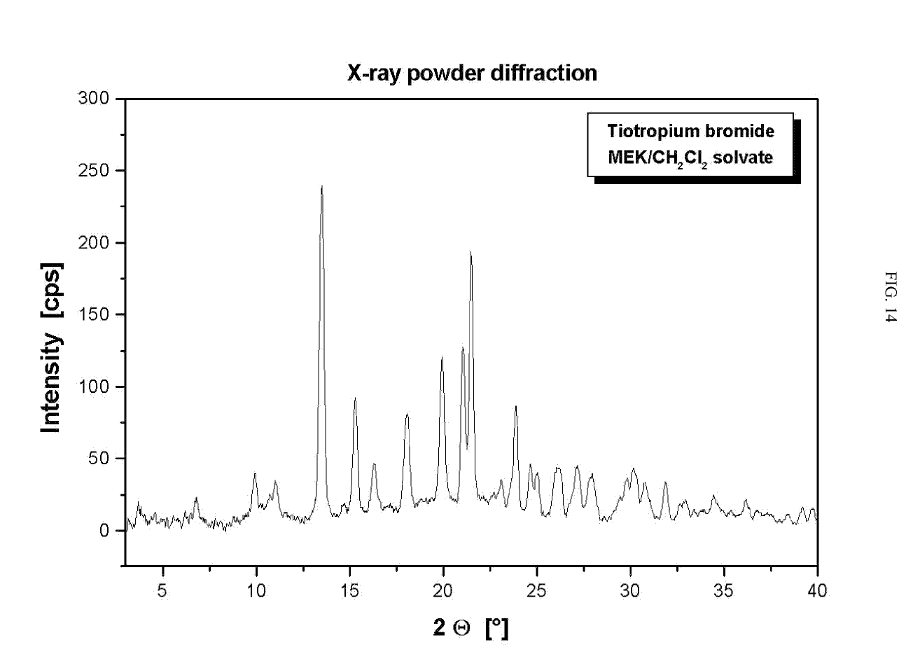

The X-ray powder diagram of the crystalline mixed methylene chloride/methyl ethyl ketone solvate of tiotropium bromide is depicted in FIG. 14.

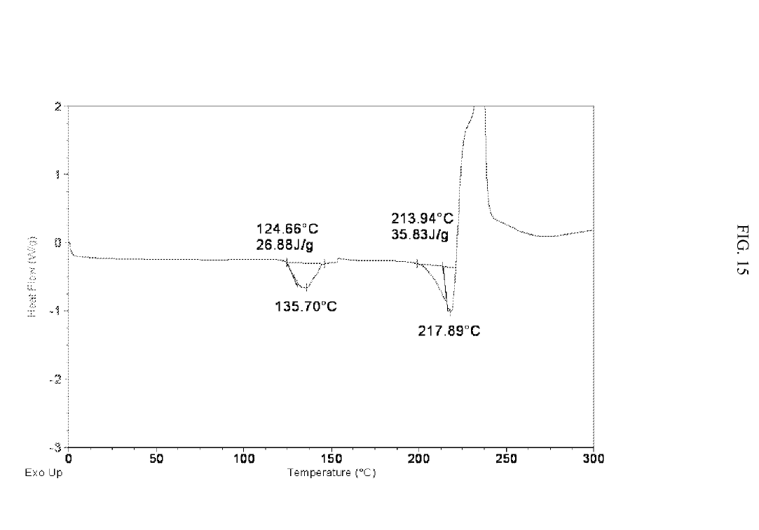

Furthermore, the crystalline mixed methylene chloride/methyl ethyl ketone solvate of tiotropium bromide according to the invention is characterised by an endothermic peak at 218° C. occurring during thermal analysis using DSC, indicating melting of tis form. An additional small endothermic event appears at 136° C. at which desolvation is observed The DSC diagram of the crystalline mixed methylene chloride/methyl ethyl ketone solvate of tiotropium bromide according to the invention is depicted in FIG. 15.

In a yet another embodiment, the present invention relates to a novel crystalline 1-butanol solvate of tiotropium bromide. In another aspect the present invention relates to a method of preparing the new crystalline 1-butanol of tiotropium bromide which is explained by way of example in the experimental section that follows.

The crystalline 1-butanol solvate of tiotropium bromide according to the invention is characterised in that in the X-ray powder diagram it has the following characteristic peaks (most dominant ones) with the values d=9.00 Å; 8.12 Å; 6.66 Å; 5.80 Å; 5.40 Å, 4.94 Å; 4.51 Å; 4.29 Å, and 4.17 Å. For more details see table 9.

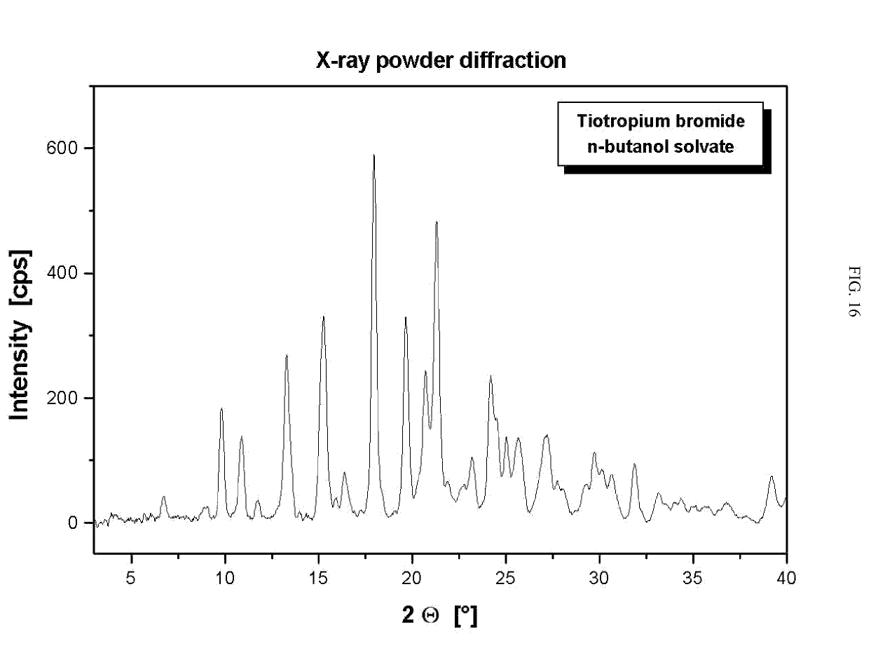

The X-ray powder diagram of the crystalline 1-butanol solvate of tiotropium bromide is depicted in FIG. 16.

A closer look to the X-ray powder diffraction patterns shows that the diagrams of the different solvates are very similar indicating that tiotropium bromide forms several solvates which are isostructural to each other.

The present invention also relates to the use of the crystalline tiotropium bromide forms according to the invention for preparing a pharmaceutical composition for the treatment of respiratory complaints, particularly for the treatment of COPD and/or asthma.

The present invention also relates to methods for the preparation of the crystalline tiotropium bromide forms according to the inventions.

The present invention relates to a method for the preparation of crystalline tiotropium bromide anhydrate according to the invention, characterized in that a solution of crystalline tiotropium bromide monohydrate in dimethylformamide is added to acetonitril, the resulting mixture being cooled to a temperature below 20° C., preferably below 10° and the resulting crystals being isolated. The present invention furthermore relates to the use of crystalline tiotropium bromide monohydrate as a starting material for the preparation of crystalline tiotropium bromide anhydrate.

The present invention also relates to a method for the preparation of crystalline methanol solvate of tiotropium bromide, characterized in that an anhydrous tiotropium bromide is recrystallized from a methanol containing solvent, preferably from a solvent mixture comprising methanol and acetone, more preferably from a solvent mixture comprising methanol, acetone and water. The present invention furthermore relates to the use of anhydrous tiotropium bromide as a starting material for the preparation of crystalline methanol solvate of tiotropium bromide.

The present invention also relates to a method for the preparation of crystalline ethanol solvate of tiotropium bromide, characterized in that an anhydrous tiotropium bromide is recrystallized from an ethanol containing solvent, preferably under heating and subsequent cooling. The present invention furthermore relates to the use of anhydrous tiotropium bromide as a starting material for the preparation of crystalline ethanol solvate of tiotropium bromide.

The present invention relates to a method for the preparation of crystalline isopropanol solvate of tiotropium bromide, characterized in that a solution of crystalline tiotropium bromide monohydrate in isopropanol is cooled to a temperature below 20° C., preferably below 10° and the resulting crystals being isolated. The present invention furthermore relates to the use of crystalline tiotropium bromide monohydrate as a starting material for the preparation of crystalline isopropanol solvate of tiotropium bromide.

The present invention relates to a method for the preparation of crystalline THF solvate of tiotropium bromide, characterized in that a solution of crystalline tiotropium bromide monohydrate in a suitable alcohol, preferably in benzyl alcohol is added to a solvent comprising THF, preferably pure THF. The present invention furthermore relates to the use of crystalline tiotropium bromide monohydrate as a starting material for the preparation of crystalline THF solvate of tiotropium bromide.

The present invention relates to a method for the preparation of crystalline 1,4-dioxane solvate of tiotropium bromide, characterized in that a solution of crystalline tiotropium bromide monohydrate in a suitable alcohol, preferably in benzyl alcohol is added to a solvent comprising 1,4-dioxane, preferably pure 1,4-dioxane. The present invention furthermore relates to the use of crystalline tiotropium bromide monohydrate as a starting material for the preparation of crystalline 1,4-dioxane solvate of tiotropium bromide.

The present invention relates to a method for the preparation of crystalline DMF solvate of tiotropium bromide, characterized in that a solution of crystalline tiotropium bromide monohydrate in DMF is added to methyl tert.-butyl ether. The present invention furthermore relates to the use of crystalline tiotropium bromide monohydrate as a starting material for the preparation of crystalline DMF solvate of tiotropium bromide.

The present invention relates to a method for the preparation of crystalline mixed methylene chloride/methyl ethyl ketone solvate of tiotropium bromide, characterized in that a solution of crystalline tiotropium bromide monohydrate in a suitable alcohol, preferably in benzyl alcohol is added to a solvent comprising methylene chloride and methyl ethyl ketone, the mixture thus obtained being optionally cooled below 20° C., preferably below 10° C. The present invention furthermore relates to the use of crystalline tiotropium bromide monohydrate as a starting material for the preparation of crystalline mixed methylene chloride/methyl ethyl ketone solvate of tiotropium bromide.

The present invention relates to a method for the preparation of crystalline 1-butanol solvate of tiotropium bromide, characterized in that a solution of crystalline tiotropium bromide monohydrate in a suitable alcohol, preferably in benzyl alcohol is added to a solvent comprising 1-butanol, preferably pure 1-butanol, the mixture thus obtained being optionally cooled below 20° C., preferably below 10° C. The present invention furthermore relates to the use of crystalline tiotropium bromide monohydrate as a starting material for the preparation of crystalline 1-butanol solvate of tiotropium bromide.

### EXAMPLES

The Examples that follow serve to illustrate the present invention still further, without restricting the scope of the invention to the embodiments by way of example that follow.

A) Examples of Synthesis of the Crystalline Forms According to the Invention

### Example 1

### Crystalline Tiotropium Bromide Anhydrate

A solution of tiotropium bromide monohydrate (obtained according to WO 02/30928) in anhydrous dimethylformamide (21 μL; 70 mg/mL) was added to anhydrous acetonitril (100 μL). The solution was cooled to 5° C. and was incubated overnight. Crystals were formed and were collected by removal of the mother liquor.

### Example 2

### Crystalline Tiotropium Bromide Anhydrate

Tiotropium bromide monohydrate (54.3 mg and obtained according to WO 02/30928) was dissolved in anhydrous dimethylformamide (0.6 mL) and added to anhydrous acetonitrile (3.0 mL). The crystallization was seeded from crystals of the above example 1. Crystals formed overnight at 5° C. and were collected by filtration. The crystalline solid was washed immediately with additional anhydrous acetonitrile (2 mL) and allowed to air dry.

### Example 3

### Crystalline Methanol Solvate of Tiotropium Bromide

Anhydrous tiotropium bromide (5.0 mg; obtainable according to WO 03/000265) was recrystallized from a methanol/acetone/water mixture (66:33:1; 50 μL). Recrystallization was induced by partial evaporation of the solution (˜25 μL) and incubation at −20° C. The solvate is also formed from recrystallization from anhydrous methanol.

### Example 4

### Crystalline Ethanol Solvate of Tiotropium Bromide

Anhydrous tiotropium bromide (50 mg; obtainable according to WO 03/000265) was recrystallized from ethanol (500 μL) by heating, then cooling and seeding with crystals of example 3.

### Example 5

### Crystalline Isopropanol Solvate of Tiotropium Bromide

A benzyl alcohol solution of tiotropium bromide monohydrate as obtained according to WO02/30928 (0.070 mL, 100 mg/ml) was added to isopropanol (1 mL, anhydrous and stored over molecular sieves) and stored at 5° C. overnight. The resulting crystals were isolated from the mother liquor.

### Example 6

### Crystalline THF Solvate of Tiotropium Bromide

A benzyl alcohol solution of tiotropium bromide monohydrate as obtained according to WO02/30928 (0.08 mL, 100 mg/ml) was dropped into tetrahydrofuran (1 mL) while stirring. The solvate formed immediately upon mixing and was collected by filtering.

### Example 7

### Crystalline 1,4-Dioxane Solvate of Tiotropium Bromide

A benzyl alcohol solution of tiotropium bromide monohydrate as obtained according to WO02/30928 (1.1 mL, 50 mg/ml) was dropped into 1,4-dioxane (5 mL) while stirring. The solvate formed, was isolated by filtration, and was allowed to air dry.

### Example 8

### Crystalline DMF Solvate of Tiotropium Bromide

A DMF solution of tiotropium bromide monohydrate as obtained according to WO02/30928 (0.15 mL, 83 mg/ml) was added to methyl tert.-butyl ether (2 mL). An amorphous solid formed and was allowed to sit for 2 days. The resulting crystalline solvate was filtered and characterized.

### Example 9

### Crystalline Mixed Methylene Chloride/Methyl Ethyl Ketone Solvate of Tiotropium Bromide

A benzyl alcohol solution of tiotropium bromide monohydrate as obtained according to WO02/30928 (0.17 mL, 90 mg/ml) was dropped into methylene chloride (0.5 mL) and methyl ethyl ketone (0.5 mL). The solution was stored at 5° C. overnight. Bulky transparent crystals of the mixed solvate formed and the excess mother liquor was removed.

### Example 10

### Crystalline 1-Butanol Solvate of Tiotropium Bromide

A benzyl alcohol solution of tiotropium bromide monohydrate as obtained according to WO02/30928 (0.17 mL, 90 mg/ml) was dropped into 1-butanol (0.5 mL) and methyl tert-butyl ether (0.5 mL). The solution was stored at 5° C. overnight. The solvate formed as a white crystalline solid which was filtered and analyzed.

B) Analytical Methods

B.1 X-Ray Powder Diffraction

X-ray powder diffraction patterns were obtained using the Rigaku D/Max Rapid X-ray Diffractometer equipped with a copper source (Cu/K_{α} 1.54056 Å), manual x-y stage, and 0.3 mm collimator. The sample was loaded into a 0.3 mm boron-rich glass capillary tube by sectioning off one end of the tube and tapping the open, sectioned end into a bed of sample. The loaded capillary was mounted in a holder that was secured into the x-y stage. A diffractogram was acquired under ambient conditions at a power setting of 46 kV at 40 mA in reflection mode, while oscillating about the omega-axis from 0-5° at 1°/sec and spinning about the phi-axis at 2°/sec. The diffractogram obtained was integrated over 2-theta from 2-40 degrees and chi (1 segment) from 0-360° at a step size of 0.02° using the cylint utility in the RINT Rapid display software provided with the instrument. The dark counts value was set to 8 as per the system calibration; normalization was set to average; the omega offset was set to 180°; and no chi or phi offsets were used for the integration. Diffraction patterns were viewed using Jade software, which was used to remove the background from the patterns and to assign peak positions.

B.2. Differential Scanning Calorimetry (DSC)

An aliquot of the sample was weighed into an aluminum hermetic sample pan, which was sealed by crimping. The sample pan was loaded into the apparatus, which is equipped with an autosampler. A thermogram was obtained by individually heating the sample at a rate of 10° C./min from Tₘᵢₙ (typically room temperature) to Tₘₐₓ (typically 350° C.) using an empty aluminum hermetic pan as a reference. Dry nitrogen was used as a sample purge gas and was set at a flow rate of 50 mL/min. Thermal transitions were viewed and analyzed using the analysis software provided with the instrument.

X-Ray Powder Diffraction Pattern of Crystalline Tiotropium Bromide Anhydrate

The tiotropium bromide anhydrate obtained by the above method is highly crystalline. It was investigated further by X-ray powder diffraction. The X-ray powder diagram obtained for the tiotropium bromide anhydrate according to the invention is shown in FIG. 1.

The following Table 1 lists the characteristic peaks and standardised intensities.

TABLE 1
X-ray powder reflections (up to 30° 2Θ) and intensities
(normalized) of anhydrous crystalline tiotropium bromide

| 2Θ [°] | d [Å] | I/Iₒ [%] |
|---|---|---|
| 8.98 | 9.84 | 18 |
| 9.94 | 8.89 | 22 |
| 10.91 | 8.10 | 24 |
| 11.73 | 7.54 | 22 |
| 12.74 | 6.94 | 1 |
| 13.41 | 6.60 | 5 |
| 15.04 | 5.89 | 100 |
| 15.86 | 5.58 | 4 |
| 16.26 | 5.45 | 8 |
| 17.34 | 5.11 | 3 |
| 18.10 | 4.90 | 47 |
| 18.30 | 4.84 | 42 |
| 19.02 | 4.66 | 7 |
| 19.58 | 4.53 | 4 |
| 20.25 | 4.38 | 9 |
| 20.49 | 4.33 | 11 |
| 20.89 | 4.25 | 22 |
| 21.27 | 4.17 | 22 |
| 21.92 | 4.05 | 61 |
| 23.13 | 3.84 | 30 |
| 23.67 | 3.76 | 12 |
| 24.12 | 3.69 | 17 |
| 24.72 | 3.60 | 11 |
| 25.28 | 3.52 | 13 |
| 25.90 | 3.44 | 16 |
| 26.52 | 3.36 | 3 |
| 26.99 | 3.30 | 5 |
| 27.66 | 3.22 | 11 |
| 28.32 | 3.15 | 8 |
| 28.74 | 3.10 | 6 |
| 29.10 | 3.07 | 10 |
| 30.05 | 2.97 | 8 |

In the above Table the value “2 Θ [°]” represents the diffraction angle in degrees and the value “d [Å]” represents the specified lattice plane intervals in Å.

X-Ray Powder Diffraction Pattern of the Crystalline Methanol Solvate of Tiotropium Bromide

The crystalline methanol solvate of tiotropium bromide obtained by the above method was investigated further by X-ray powder diffraction. The X-ray powder diagram obtained for the crystalline methanol solvate of tiotropium bromide according to the invention is shown in FIG. 3. The following Table 2 lists the characteristic peaks and standardised intensities.

TABLE 2
X-ray powder reflections (up to 30° 2Θ) and intensities
(normalized) of a solvated form of tiotropium bromide
containing methanol with a stoichiometry of tiotropium
bromide:methanol close to 1:1

| 2Θ [°] | d [Å] | I/Iₒ [%] |
|---|---|---|
| 6.79 | 13.01 | 6 |
| 9.82 | 9.00 | 32 |
| 10.91 | 8.10 | 24 |
| 12.88 | 6.87 | 7 |
| 13.45 | 6.58 | 58 |
| 14.29 | 6.19 | 2 |
| 15.34 | 5.77 | 59 |
| 16.55 | 5.35 | 16 |
| 17.93 | 4.94 | 75 |
| 19.71 | 4.50 | 74 |
| 20.44 | 4.34 | 10 |
| 20.90 | 4.25 | 33 |
| 21.45 | 4.14 | 100 |
| 22.61 | 3.93 | 12 |
| 23.10 | 3.85 | 13 |
| 23.53 | 3.78 | 6 |
| 24.22 | 3.67 | 27 |
| 24.54 | 3.63 | 27 |
| 25.05 | 3.55 | 15 |
| 25.50 | 3.49 | 12 |
| 25.85 | 3.44 | 8 |
| 26.10 | 3.41 | 14 |
| 27.20 | 3.28 | 23 |
| 27.99 | 3.19 | 12 |
| 28.27 | 3.15 | 10 |
| 28.85 | 3.09 | 7 |
| 29.30 | 3.05 | 13 |
| 29.70 | 3.01 | 26 |
| 30.25 | 2.95 | 10 |

X-Ray Powder Diffraction Pattern of the Crystalline Ethanol Solvate of Tiotropium Bromide

The crystalline ethanol solvate of tiotropium bromide obtained by the above method was investigated further by X-ray powder diffraction. The X-ray powder diagram obtained for the crystalline ethanol solvate of tiotropium bromide according to the invention is shown in FIG. 5. The following Table 3 lists the characteristic peaks and standardised intensities.

TABLE 3
X-ray powder reflections (up to 30° 2Θ) and intensities
(normalized) of a solvated form of tiotropium bromide
containing ethanol with a stoichiometry
of tiotropium bromide:ethanol close to 2:1

| 2Θ [°] | d [Å] | I/Iₒ [%] |
|---|---|---|
| 6.69 | 13.20 | 4 |
| 9.92 | 8.91 | 36 |
| 11.03 | 8.01 | 32 |
| 12.81 | 6.90 | 6 |
| 13.41 | 6.60 | 91 |
| 14.72 | 6.01 | 6 |
| 15.31 | 5.78 | 77 |
| 16.32 | 5.43 | 20 |
| 18.10 | 4.90 | 91 |
| 19.91 | 4.46 | 94 |
| 20.94 | 4.24 | 44 |
| 21.41 | 4.15 | 100 |
| 22.34 | 3.98 | 6 |
| 23.13 | 3.84 | 15 |
| 23.65 | 3.76 | 26 |
| 23.99 | 3.71 | 25 |
| 24.68 | 3.60 | 30 |
| 25.09 | 3.55 | 31 |
| 26.01 | 3.42 | 43 |
| 27.08 | 3.29 | 38 |
| 27.88 | 3.20 | 27 |
| 29.15 | 3.06 | 6 |
| 29.65 | 3.01 | 17 |
| 30.18 | 2.96 | 21 |

X-Ray Powder Diffraction Pattern of the Crystalline Isopropanol Solvate of Tiotropium Bromide

The crystalline isopropanol solvate of tiotropium bromide obtained by the above method was investigated further by X-ray powder diffraction. The X-ray powder diagram obtained for the crystalline isopropanol solvate of tiotropium bromide according to the invention is shown in FIG. 7. The following Table 4 lists the characteristic peaks and standardised intensities.

TABLE 4
X-ray powder reflections (up to 30° 2Θ) and
intensities (normalized) of a solvated form of tiotropium
bromide containing isopropanol with a
stoichiometry of tiotropium bromide:isopropanol close to 2:1

| 2Θ [°] | d [Å] | I/Iₒ [%] |
|---|---|---|
| 6.73 | 13.12 | 7 |
| 9.86 | 8.96 | 28 |
| 10.97 | 8.06 | 26 |
| 13.28 | 6.66 | 55 |
| 15.28 | 5.80 | 65 |
| 16.22 | 5.46 | 18 |
| 18.04 | 4.91 | 91 |
| 19.80 | 4.48 | 71 |
| 20.71 | 4.28 | 48 |
| 21.26 | 4.17 | 100 |
| 22.35 | 3.98 | 7 |
| 23.02 | 3.86 | 11 |
| 23.55 | 3.77 | 23 |
| 24.00 | 3.71 | 22 |
| 24.59 | 3.62 | 21 |
| 25.08 | 3.55 | 24 |
| 25.82 | 3.45 | 28 |
| 27.00 | 3.30 | 19 |
| 27.66 | 3.22 | 18 |
| 29.55 | 3.02 | 13 |
| 29.85 | 2.99 | 17 |
| 30.22 | 2.96 | 16 |
| 30.69 | 2.91 | 16 |

X-Ray Powder Diffraction Pattern of the Crystalline THF Solvate of Tiotropium Bromide

The crystalline THF solvate of tiotropium bromide obtained by the above method was investigated further by X-ray powder diffraction. The X-ray powder diagram obtained for the crystalline THF solvate of tiotropium bromide according to the invention is shown in FIG. 9. The following Table 5 lists the characteristic peaks and standardised intensities.

TABLE 5
X-ray powder reflections (up to 30° 2Θ) and intensities
(normalized) of a solvated form of tiotropium bromide
containing tetrahydrofurane (=THF)
with a stoichiometry of tiotropium bromide:THF close to 2:1

| 2Θ [°] | d [Å] | I/Iₒ [%] |
|---|---|---|
| 6.72 | 13.15 | 5 |
| 9.85 | 8.97 | 27 |
| 10.65 | 8.30 | 9 |
| 11.02 | 8.03 | 14 |
| 13.03 | 6.79 | 14 |
| 13.41 | 6.60 | 47 |
| 15.28 | 5.80 | 77 |
| 16.26 | 5.45 | 12 |
| 16.86 | 5.25 | 8 |
| 18.02 | 4.92 | 100 |
| 19.82 | 4.48 | 55 |
| 20.64 | 4.30 | 49 |
| 20.87 | 4.25 | 46 |
| 21.41 | 4.15 | 98 |
| 22.46 | 3.96 | 9 |
| 22.94 | 3.87 | 14 |
| 23.58 | 3.77 | 15 |
| 23.97 | 3.71 | 32 |
| 24.52 | 3.63 | 21 |
| 25.03 | 3.56 | 29 |
| 25.92 | 3.43 | 22 |
| 27.04 | 3.29 | 23 |
| 27.83 | 3.20 | 16 |
| 28.77 | 3.10 | 5 |
| 29.64 | 3.01 | 22 |
| 30.05 | 2.97 | 16 |

X-Ray Powder Diffraction Pattern of the Crystalline 1,4-Dioxane Solvate of Tiotropium Bromide

The crystalline 1,4-dioxane solvate of tiotropium bromide obtained by the above method was investigated further by X-ray powder diffraction. The X-ray powder diagram obtained for the crystalline 1,4-dioxane solvate of tiotropium bromide according to the invention is shown in FIG. 11. The following Table 6 lists the characteristic peaks and standardised intensities.

TABLE 6
X-ray powder reflections (up to 30° 2Θ) and
intensities (normalized) of a solvated form of tiotropium
bromide containing dioxane with a stoichiometry
of tiotropium bromide:dioxane close to 2:1

| 2Θ [°] | d [Å] | I/Iₒ [%] |
|---|---|---|
| 6.69 | 13.20 | 3 |
| 9.91 | 8.92 | 15 |
| 10.95 | 8.08 | 9 |
| 13.42 | 6.59 | 49 |
| 14.56 | 6.08 | 3 |
| 15.29 | 5.79 | 51 |
| 16.39 | 5.41 | 13 |
| 18.01 | 4.92 | 80 |
| 19.68 | 4.51 | 38 |
| 20.00 | 4.44 | 34 |
| 20.80 | 4.27 | 30 |
| 21.39 | 4.15 | 100 |
| 22.81 | 3.90 | 10 |
| 23.15 | 3.84 | 7 |
| 23.97 | 3.71 | 27 |
| 24.33 | 3.66 | 12 |
| 24.84 | 3.58 | 25 |
| 25.57 | 3.48 | 11 |
| 26.11 | 3.41 | 14 |
| 27.07 | 3.29 | 24 |
| 27.95 | 3.19 | 18 |
| 29.26 | 3.05 | 8 |
| 29.85 | 2.99 | 18 |
| 30.19 | 2.96 | 20 |

X-Ray Powder Diffraction Pattern of the Crystalline DMF Solvate of Tiotropium Bromide

The crystalline DMF solvate of tiotropium bromide obtained by the above method was investigated further by X-ray powder diffraction. The X-ray powder diagram obtained for the crystalline DMF solvate of tiotropium bromide according to the invention is shown in FIG. 13. The following Table 7 lists the characteristic peaks and standardised intensities.

TABLE 7
X-ray powder reflections (up to 30° 2Θ) and
intensities (normalized) of a solvated form of tiotropium
bromide containing N,N-dimethylformamide (=DMF)
with a stoichiometry of tiotropium bromide:DMF close to 2:1

| 2Θ [°] | d [Å] | I/Iₒ [%] |
|---|---|---|
| 6.83 | 12.93 | 4 |
| 8.81 | 10.03 | 45 |
| 9.88 | 8.95 | 28 |
| 11.02 | 8.02 | 28 |
| 11.73 | 7.54 | 51 |
| 12.96 | 6.82 | 32 |
| 13.51 | 6.55 | 53 |
| 14.00 | 6.32 | 41 |
| 15.31 | 5.78 | 70 |
| 15.57 | 5.69 | 100 |
| 16.40 | 5.40 | 21 |
| 17.24 | 5.14 | 23 |
| 17.71 | 5.00 | 77 |
| 17.95 | 4.94 | 99 |
| 19.79 | 4.48 | 78 |
| 20.27 | 4.38 | 62 |
| 21.07 | 4.21 | 58 |
| 21.59 | 4.11 | 99 |
| 22.23 | 4.00 | 46 |
| 22.83 | 3.89 | 27 |
| 23.34 | 3.81 | 44 |
| 24.09 | 3.69 | 48 |
| 24.72 | 3.60 | 37 |
| 25.01 | 3.56 | 31 |
| 25.80 | 3.45 | 32 |
| 26.04 | 3.42 | 35 |
| 27.01 | 3.30 | 68 |
| 27.95 | 3.19 | 23 |
| 29.11 | 3.07 | 18 |
| 29.48 | 3.03 | 14 |
| 29.90 | 2.99 | 23 |

X-Ray Powder Diffraction Pattern of the Crystalline Mixed Methylene Chloride/Methyl Ethyl Ketone Solvate of Tiotropium Bromide

The crystalline mixed methylene chloride/methyl ethyl ketone solvate of tiotropium bromide obtained by the above method was investigated further by X-ray powder diffraction. The X-ray powder diagram obtained for the crystalline mixed methylene chloride/methyl ethyl ketone solvate of tiotropium bromide according to the invention is shown in FIG. 14. The following Table 8 lists the characteristic peaks and standardised intensities.

TABLE 8
X-ray powder reflections (up to 30° 2Θ) and intensities
(normalized) of a solvated form of tiotropium bromide
containing methylethyl ketone (=MEK)
and dichloromethane (CH₂Cl₂)

| 2Θ [°] | d [Å] | I/Iₒ [%] |
|---|---|---|
| 6.79 | 13.01 | 10 |
| 9.92 | 8.91 | 17 |
| 11.03 | 8.02 | 15 |
| 13.49 | 6.56 | 100 |
| 15.30 | 5.79 | 38 |
| 16.30 | 5.43 | 20 |
| 18.04 | 4.91 | 34 |
| 19.93 | 4.45 | 50 |
| 21.05 | 4.22 | 53 |
| 21.49 | 4.13 | 81 |
| 23.08 | 3.85 | 15 |
| 23.87 | 3.72 | 36 |
| 24.65 | 3.61 | 20 |
| 25.00 | 3.56 | 17 |
| 26.17 | 3.40 | 18 |
| 27.16 | 3.28 | 19 |
| 27.90 | 3.20 | 16 |
| 29.42 | 3.03 | 10 |
| 29.79 | 3.00 | 15 |
| 30.17 | 2.96 | 18 |

X-Ray Powder Diffraction Pattern of the Crystalline 1-Butanol Solvate of Tiotropium Bromide

The crystalline 1-butanol solvate of tiotropium bromide obtained by the above method was investigated further by X-ray powder diffraction. The X-ray powder diagram obtained for the crystalline 1-butanol solvate of tiotropium bromide according to the invention is shown in FIG. 16. The following Table 9 lists the characteristic peaks and standardised intensities.

TABLE 9
X-ray powder reflections (up to 30° 2Θ) and intensities (normalized) of
a solvated form of tiotropium bromide containing n-butanol with a
stoichiometry of tiotropium bromide:n-butanol close to 2:1

| 2Θ [°] | d [Å] | I/Iₒ [%] |
|---|---|---|
| 6.72 | 13.14 | 7 |
| 8.90 | 9.93 | 4 |
| 9.82 | 9.00 | 31 |
| 10.88 | 8.12 | 24 |
| 11.73 | 7.54 | 6 |
| 13.28 | 6.66 | 46 |
| 15.27 | 5.80 | 56 |
| 16.39 | 5.40 | 14 |
| 17.96 | 4.94 | 100 |
| 19.67 | 4.51 | 56 |
| 20.71 | 4.29 | 41 |
| 21.30 | 4.17 | 82 |
| 21.89 | 4.06 | 11 |
| 22.76 | 3.90 | 10 |
| 23.19 | 3.83 | 18 |
| 24.19 | 3.68 | 40 |
| 24.49 | 3.63 | 29 |
| 25.03 | 3.55 | 23 |
| 25.66 | 3.47 | 23 |
| 27.17 | 3.28 | 24 |
| 27.73 | 3.21 | 12 |
| 28.04 | 3.18 | 9 |
| 29.27 | 3.05 | 11 |
| 29.70 | 3.01 | 19 |
| 30.14 | 2.96 | 15 |

C: Formulations Containing the Tiotropium Bromide Forms According to the Invention

The crystalline tiotropium bromide forms according to the invention are particularly well suited to the preparation of, for example, pharmaceutical formulations for administration by inhalation such as inhalable powders or for example propellant-containing aerosol formulations, particularly inhalable powders and propellant-containing aerosol suspensions. These pharmaceutical formulations or compositions may contain in addition to the crystalline tiotropium forms according to the invention one or more additional active ingredients selected from among betamimetics, EGFR inhibitors, PDEIV-inhibitors, steroids, and LTD4 antagonists, optionally together with a pharmaceutically acceptable excipient.

C.1: Inhalable Powders

The present invention also relates to inhalable powder containing 0.001 to 3% tiotropium in the form of the crystalline tiotropium bromide forms according to the invention combined with a physiologically acceptable excipient. By tiotropium is meant the ammonium cation.

Inhalable powders which contain 0.01 to 2% tiotropium are preferred according to the invention. Particularly preferred inhalable powders contain tiotropium in an amount from about 0.03 to 1%, preferably 0.05 to 0.6%, particularly preferably 0.06 to 0.3%. Of particular importance according to the invention, finally, are inhalable powders which contain about 0.08 to 0.22% tiotropium.

The amounts of tiotropium specified above are based on the amount of tiotropium cation contained.

The excipients that are used for the purposes of the present invention are prepared by suitable grinding and/or screening using current methods known in the art. The excipients used according to the invention may also be mixtures of excipients which are obtained by mixing excipient fractions of different mean particle sizes.

Examples of physiologically acceptable excipients which may be used to prepare the inhalable powders for use in the inhalettes according to the invention include monosaccharides (e.g. glucose, fructose or arabinose), disaccharides (e.g. lactose, saccharose, maltose, trehalose), oligo- and polysaccharides (e.g. dextrans, dextrins, maltodextrin, starch, cellulose), polyalcohols (e.g. sorbitol, mannitol, xylitol), cyclodextrins (e.g. α-cyclodextrin, β-cyclodextrin, χ-cyclodextrin, methyl-β-cyclodextrin, hydroxypropyl-β-cyclodextrin), amino acids (e.g. arginine hydrochloride) or salts (e.g. sodium chloride, calcium carbonate), or mixtures thereof. Preferably, mono- or disaccharides are used, while the use of lactose or glucose is preferred, particularly, but not exclusively, in the form of their hydrates. For the purposes of the invention, lactose is the particularly preferred excipient.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. The average particle size may be determined using methods known in the art (cf. for example WO 02/30389, paragraphs A and C). Finally, in order to prepare the inhalable powders according to the invention, micronised crystalline tiotropium bromide anhydrate, which is preferably characterised by an average particle size of 0.5 to 10 μm, particularly preferably from 1 to 5 μm, is added to the excipient mixture (cf. for example WO 02/30389, paragraph B). Processes for grinding and micronising active substances are known from the prior art.

If no specifically prepared excipient mixture is used as the excipient, it is particularly preferable to use excipients which have a mean particle size of 10-50 μm and a 10% fine content of 0.5 to 6 μm.

By average particle size is meant here the 50% value of the volume distribution measured with a laser diffractometer using the dry dispersion method. The average particle size may be determined using methods known in the art (cf. for example WO 02/30389, paragraphs A and C). Analogously, the 10% fine content in this instance refers to the 10% value of the volume distribution measured using a laser diffractometer. In other words, for the purposes of the present invention, the 10% fine content denotes the particle size below which 10% of the quantity of particles is found (based on the volume distribution).

The percentages given within the scope of the present invention are always percent by weight, unless specifically stated to the contrary.

In particularly preferred inhalable powders the excipient is characterised by a mean particle size of 12 to 35 μm, particularly preferably from 13 to 30 μm.

Also particularly preferred are those inhalable powders wherein the 10% fine content is about 1 to 4 μm, preferably about 1.5 to 3 μm.

The inhalable powders according to the invention are characterised, in accordance with the problem on which the invention is based, by a high degree of homogeneity in the sense of the accuracy of single doses. This is in the region of <8%, preferably <6%, most preferably <4%.

After the starting materials have been weighed out the inhalable powders are prepared from the excipient and the active substance using methods known in the art. Reference may be made to the disclosure of WO 02/30390, for example. The inhalable powders according to the invention may accordingly be obtained by the method described below, for example. In the preparation methods described hereinafter the components are used in the proportions by weight described in the above-mentioned compositions of the inhalable powders.

First, the excipient and the active substance are placed in a suitable mixing container. The active substance used has an average particle size of 0.5 to 10 μm, preferably 1 to 6 μm, most preferably 2 to 5 μm. The excipient and the active substance are preferably added using a sieve or a granulating sieve with a mesh size of 0.1 to 2 mm, preferably 0.3 to 1 mm, most preferably 0.3 to 0.6 mm. Preferably, the excipient is put in first and then the active substance is added to the mixing container. During this mixing process the two components are preferably added in batches. It is particularly preferred to sieve in the two components in alternate layers. The mixing of the excipient with the active substance may take place while the two components are still being added. Preferably, however, mixing is only done once the two components have been sieved in layer by layer.

The present invention also relates to the use of the inhalable powders according to the invention for preparing a pharmaceutical composition for the treatment of respiratory complaints, particularly for the treatment of COPD and/or asthma.

The inhalable powders according to the invention may for example be administered using inhalers which meter a single dose from a reservoir by means of a measuring chamber (e.g. according to U.S. Pat. No. 4,570,630A) or by other means (e.g. according to DE 36 25 685 A). Preferably, however, the inhalable powders according to the invention are packed into capsules (to make so-called inhalettes), which are used in inhalers such as those described in WO 94/28958, for example.

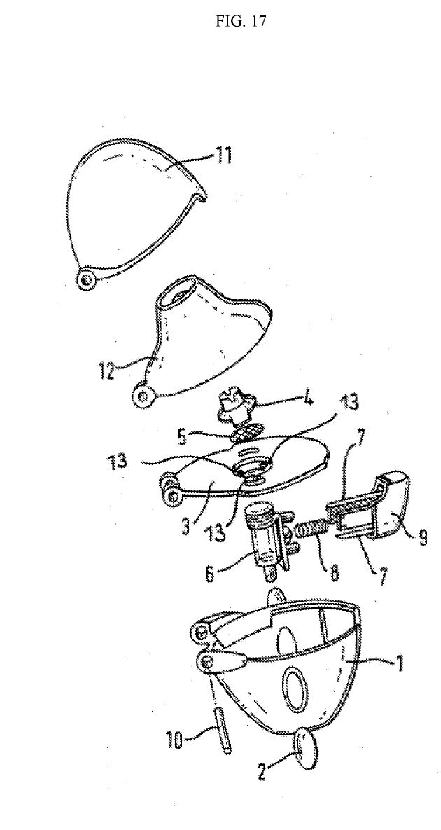

Most preferably, the capsules containing the inhalable powder according to the invention are administered using an inhaler as shown in FIG. 17. This inhaler is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut and airholes 13 for adjusting the flow resistance.

The present invention further relates to the use of the inhalable powders containing one or several, preferably one of the crystalline tiotropium bromide forms according to the invention for preparing a pharmaceutical composition for treating respiratory complaints, particularly for the treatment of COPD and/or asthma, characterised in that the inhaler described above and shown in FIG. 17 is used.

For administering the inhalable powders containing the crystalline tiotropium bromide forms according to the invention using powder-filled capsules it is particularly preferred to use capsules the material of which is selected from among the synthetic plastics, most preferably selected from among polyethylene, polycarbonate, polyester, polypropylene and polyethylene terephthalate. Particularly preferred synthetic plastic materials are polyethylene, polycarbonate or polyethylene terephthalate. If polyethylene is used as one of the capsule materials which is particularly preferred according to the invention, it is preferable to use polyethylene with a density of between 900 and 1000 kg/m³, preferably 940-980 kg/m³, more preferably about 960-970 kg/m³ (high density polyethylene). The synthetic plastics according to the invention may be processed in various ways using manufacturing methods known in the art. Injection moulding of the plastics is preferred according to the invention. Injection moulding without the use of mould release agents is particularly preferred. This method of production is well defined and is characterised by being particularly reproducible.

In another aspect the present invention relates to the abovementioned capsules which contain the abovementioned inhalable powder according to the invention. These capsules may contain about 1 to 20 mg, preferably about 3 to 15 mg, most preferably about 4 to 12 mg of inhalable powder. Preferred formulations according to the invention contain 4 to 6 mg of inhalable powder. Of equivalent importance according to the invention are capsules for inhalation which contain the formulations according to the invention in an amount of from 8 to 12 mg.

The present invention also relates to an inhalation kit consisting of one or more of the above capsules characterised by a content of inhalable powder according to the invention in conjunction with the inhaler according to FIG. 17.

The present invention also relates to the use of the abovementioned capsules characterised by a content of inhalable powder according to the invention, for preparing a pharmaceutical composition for treating respiratory complaints, especially for treating COPD and/or asthma.

Filled capsules which contain the inhalable powders according to the invention are produced by methods known in the art, by filling the empty capsules with the inhalable powders according to the invention.

C.1.1: Examples of Inhalable Powders According to the Invention

The following Examples serve to illustrate the present invention in more detail without restricting the scope of the invention to the exemplifying embodiments that follow.

Active Substance

The crystalline tiotropium bromide forms according to the invention are used to produce the inhalable powders according to the invention. The micronisation of these forms may be carried out analogously to methods known in the art (cf for example WO 03/078429 A1). Where reference is made within the scope of the present invention to the mean particle size of the crystalline tiotropium bromide forms according to the invention, this is determined using methods of measurement known in the art (cf for example WO 03/078429 A1, para. D.2).

Excipient:

In the Examples that follow lactose-monohydrate is used as excipient. It may be obtained for example from Borculo Domo Ingredients, Borculo/NL under the product name Lactochem Extra Fine Powder. The specifications according to the invention for the particle size and specific surface area are met by this grade of lactose. For example, in the Examples that follow, batches of lactose were used having the following specifications:

Preparation of the Powder Formulations:

Apparatus

The following machines and equipment, for example, may be used to prepare the inhalable powders:

Mixing container or powder mixer: Turbulamischer 2 L, Type 2C; made by Willy A. Bachofen AG, CH-4500 Basel

Hand-held screen: 0.135 mm mesh size

The empty inhalation capsules may be filled with inhalable powders containing tiotropium by hand or mechanically. The following equipment may be used.

Capsule filling machine:

MG2, Type G100, manufacturer: MG2 S.r.1, I-40065 Pian di Macina di Pianoro (BO), Italy

### FORMULATION EXAMPLES

### Formulation Example 1

### Powder Mixture

To prepare the powder mixture, 299.39 g of excipient and 0.61 g of micronised crystalline tiotropium bromide anhydrate are used.

About 40-45 g of excipient are placed in a suitable mixing container through a hand-held screen with a mesh size of 0.315 mm. Then crystalline tiotropium bromide anhydrate in batches of about 90-110 mg and excipient in batches of about 40-45 g are screened in in alternate layers. The excipient and active substance are added in 7 and 6 layers, respectively.

Having been screened in, the ingredients are then mixed (mixing speed 900 rpm). The final mixture is passed twice more through a hand-held screen and then mixed again at 900 rpm.

Using the method described in formulation Example 1 it is possible to obtain inhalable powders which when packed into suitable plastic capsules may be used to produce the following capsules for inhalation, for example:

### Formulation Example 2

| | |
|---|---|
| tiotropium bromide anhydrate: | 0.0113 mg |
| lactose monohydrate: | 5.4887 mg |
| capsule: | 100.0 mg |
| Total: | 105.5 mg |

### Formulation Example 3

| | |
|---|---|
| tiotropium bromide anhydrate: | 0.0225 mg |
| lactose monohydrate: | 5.4775 mg |
| polyethylene capsules: | 100.0 mg |
| Total: | 105.5 mg |

### Formulation Example 4

| | |
|---|---|
| tiotropium bromide anhydrate: | 0.0056 mg |
| lactose monohydrate: | 5.4944 mg |
| polyethylene capsules: | 100.0 mg |
| Total: | 105.5 mg |

### Formulation Example 5

| | |
|---|---|
| tiotropium bromide anhydrate: | 0.0113 mg |
| lactose monohydrate:* | 5.4887 mg |
| capsule: | 100.0 mg |
| Total: | 105.5 mg |

*the lactose contains 5% specifically added fine content of micronised lactose monohydrate with a mean particle size of about 4 μm.

### Formulation Example 6

| | |
|---|---|
| tiotropium bromide anhydrate: | 0.0225 mg |
| lactose monohydrate:* | 5.4775 mg |
| polyethylene capsules: | 100.0 mg |
| Total: | 105.5 mg |

*the lactose contains 5% specifically added fine content of micronised lactose monohydrate with a mean particle size of about 4 μm.

### Formulation Example 7

| | |
|---|---|
| tiotropium bromide anhydrate: | 0.0056 mg |
| lactose monohydrate:* | 5.4944 mg |
| polyethylene capsules: | 100.0 mg |
| Total: | 105.5 mg |

*the lactose contains 5% specifically added fine content of micronised lactose monohydrate with a mean particle size of about 4 μm.

It is apparent for the person of ordinary skill in the art, that the foregoing examples can be applied in analogy with one of the other crystalline forms of tiotropium bromide specified hereinbefore. In order to obtain products comprising one of the other solvates according to the invention the powder mixture according to formulation example 1 and also formulation examples 2 to 7 can easily be obtained by using one of the other crystalline solvates according to the invention instead of the tiotropium bromide anhydrate.

C.2: Propellant-Containing Aerosol Suspensions

The crystalline tiotropium bromide forms according to the invention may optionally also be administered in the form of propellant-containing inhalable aerosols. Aerosol suspensions are particularly suitable for this.

The present invention therefore also relates to suspensions of the crystalline tiotropium bromide forms according to the invention in the propellent gases HFA 227 and/or HFA 134a, optionally combined with one or more other propellent gases, preferably selected from the group consisting of propane, butane, pentane, dimethylether, CHClF₂, CH₂F₂, CF₃CH₃, isobutane, isopentane and neopentane.

According to the invention those suspensions which contain as propellent gas only HFA 227, a mixture of HFA 227 and HFA 134a or only HFA 134a are preferred. If a mixture of the propellent gases HFA 227 and HFA 134a is used in the suspension formulations according to the invention, the weight ratios in which these two propellent gas components are used are freely variable.

If one or more other propellent gases, selected from the group consisting of propane, butane, pentane, dimethylether, CHClF₂, CH₂F₂, CF₃CH₃, isobutane, isopentane and neopentane are used in addition to the propellent gases HFA 227 and/or HFA 134a in the suspension formulations according to the invention, the amount of this additional propellent gas component is preferably less than 50%, preferably less than 40%, particularly preferably less than 30%.

The suspensions according to the invention preferably contain an amount of tiotropium bromide form such that the amount of tiotropium cation is between 0.001 and 0.8%, preferably between 0.08 and 0.5%, and particularly preferably between 0.2 and 0.4% according to the invention.

Unless stated to the contrary, the percentages given within the scope of the present invention are always percent by weight.

In some cases, the term suspension formulation is used within the scope of the present invention instead of the term suspension. The two terms are to be regarded as equivalent within the scope of the present invention.

The propellant-containing inhalable aerosols or suspension formulations according to the invention may also contain other constituents such as surface-active agents (surfactants), adjuvants, antioxidants or flavourings.

The surface-active agents (surfactants) optionally present in the suspensions according to the invention are preferably selected from the group consisting of Polysorbate 20, Polysorbate 80, Myvacet 9-45, Myvacet 9-08, isopropyl myristate, oleic acid, propyleneglycol, polyethyleneglycol, Brij, ethyl oleate, glyceryl trioleate, glyceryl monolaurate, glyceryl monooleate, glyceryl monostearate, glyceryl monoricinoleate, cetylalcohol, sterylalcohol, cetylpyridinium chloride, block polymers, natural oil, ethanol and isopropanol. Of the above-mentioned suspension adjuvants Polysorbate 20, Polysorbate 80, Myvacet 9-45, Myvacet 9-08 or isopropyl myristate are preferably used. Myvacet 9-45 or isopropyl myristate are most preferably used.

If the suspensions according to the invention contain surfactants these are preferably used in an amount of 0.0005-1%, particularly preferably 0.005-0.5%.

The adjuvants optionally contained in the suspensions according to the invention are preferably selected from the group consisting of alanine, albumin, ascorbic acid, aspartame, betaine, cysteine, phosphoric acid, nitric acid, hydrochloric acid, sulphuric acid and citric acid. Ascorbic acid, phosphoric acid, hydrochloric acid or citric acid are preferably used, while hydrochloric acid or citric acid is most preferably used.

If adjuvants are present in the suspensions according to the invention, these are preferably used in an amount of 0.0001-1.0%, preferably 0.0005-0.1%, particularly preferably 0.001-0.01%, while an amount of 0.001-0.005% is particularly important according to the invention.

The antioxidants optionally contained in the suspensions according to the invention are preferably selected from the group consisting of ascorbic acid, citric acid, sodium edetate, editic acid, tocopherols, butylhydroxytoluene, butylhydroxyanisol and ascorbylpalmitate, while tocopherols, butylhydroxytoluene, butylhydroxyanisol or ascorbylpalmitate are preferably used.

The flavourings optionally contained in the suspensions according to the invention are preferably selected from the group consisting of peppermint, saccharine, Dentomint, aspartame and ethereal oils (for example cinnamon, aniseed, menthol, camphor), of which peppermint or Dentomint® are particularly preferred.

With a view to administration by inhalation it is essential to provide the active substances in finely divided form. For this purpose, the crystalline tiotropium bromide forms according to the invention are obtained in finely divided form using methods known in the prior art. Methods of micronising active substances are known in the art. Preferably after micronising the active substance has a mean particle size of 0.5 to 10 μm, preferably 1 to 6 μm, particularly preferably 1.5 to 5 μm. Preferably at least 50%, preferably at least 60%, particularly preferably at least 70% of the particles of active substance have a particle size which is within the size ranges mentioned above. Particularly preferably at least 80%, most preferably at least 90% of the particles of active substance have a particle size which is within the size ranges mentioned above.

In another aspect the present invention relates to suspensions which contain only one of the two active substances according to the invention without any other additives.

The suspensions according to the invention may be prepared using methods known in the art. For this, the constituents of the formulation are mixed with the propellent gas or gases (optionally at low temperatures) and filled into suitable containers.

The above-mentioned propellant-containing suspensions according to the invention may be administered using inhalers known in the art (pMDIs=pressurized metered dose inhalers). Accordingly, in another aspect, the present invention relates to pharmaceutical compositions in the form of suspensions as hereinbefore described combined with one or more inhalers suitable for administering these suspensions. Moreover the present invention relates to inhalers, characterised in that they contain the propellant-containing suspensions according to the invention described hereinbefore.

The present invention also relates to containers (cartridges) which when fitted with a suitable valve can be used in a suitable inhaler and which contain one of the above-mentioned propellant-containing suspensions according to the invention. Suitable containers (cartridges) and processes for filling these cartridges with the propellant-containing suspensions according to the invention are known in the art.

In view of the pharmaceutical activity of tiotropium the present invention also relates to the use of the suspensions according to the invention for preparing a pharmaceutical composition for inhalation or nasal administration, preferably for preparing a pharmaceutical composition for inhalative or nasal treatment of diseases in which anticholinergics may develop a therapeutic benefit.

Particularly preferably the present invention also relates to the use of the suspensions according to the invention for preparing a pharmaceutical composition for the inhalative treatment of respiratory complaints, preferably asthma or COPD.

The Examples that follow serve to illustrate the present invention in more detail, by way of example, without restricting it to their contents.

Examples of Aerosol Suspension Formulations

Suspensions containing other ingredients in addition to active substance and propellent gas:

### Formulation Example 8

| constituents | concentration [% w/w] |
|---|---|
| tiotropium bromide anhydrate | 0.04 |
| oleic acid | 0.005 |
| HFA-227 | 99.955 |

### Formulation Example 9

| constituents | concentration [% w/w] |
|---|---|
| tiotropium bromide anhydrate | 0.02 |
| oleic acid | 0.01 |
| HFA-227 | 60.00 |
| HFA-134a | 39.97 |

### Formulation Example 10

| constituents | concentration [% w/w] |
|---|---|
| tiotropium bromide anhydrate | 0.02 |
| isopropylmyristate | 1.00 |
| HFA-227 | 98.98 |

### Formulation Example 11

| constituents | concentration [% w/w] |
|---|---|
| tiotropium bromide anhydrate | 0.02 |
| Myvacet 9-45 | 0.3 |
| HFA-227 | 99.68 |

### Formulation Example 12

| constituents | concentration [% w/w] |
|---|---|
| tiotropium bromide anhydrate | 0.02 |
| Myvacet 9-45 | 0.1 |
| HFA-227 | 60.00 |
| HFA-134a | 39.88 |

### Formulation Example 13

| constituents | concentration [% w/w] |
|---|---|
| tiotropium bromide anhydrate | 0.04 |
| Polysorbate 80 | 0.04 |
| HFA-227 | 99.92 |

### Formulation Example 14

| constituents | concentration [% w/w] |
|---|---|
| tiotropium bromide anhydrate | 0.01 |
| Polysorbate 20 | 0.20 |
| HFA-227 | 99.78 |

### Formulation Example 15

| constituents | concentration [% w/w] |
|---|---|
| tiotropium bromide anhydrate | 0.04 |
| Myvacet 9-08 | 01.00 |
| HFA-227 | 98.96 |

### Formulation Example 16

| constituents | concentration [% w/w] |
|---|---|
| tiotropium bromide anhydrate | 0.02 |
| isopropylmyristate | 0.30 |
| HFA-227 | 20.00 |
| HFA-134a | 79.68 |

Suspensions containing only active substance and propellent gas:

### Formulation Example 17

| constituents | concentration [% w/w] |
|---|---|
| tiotropium bromide anhydrate | 0.02 |
| HFA-227 | 60.00 |
| HFA-134a | 39.98 |

### Formulation Example 18

| constituents | concentration [% w/w] |
|---|---|
| tiotropium bromide anhydrate | 0.02 |
| HFA-227 | 99.98 |

### Formulation Example 19

| constituents | concentration [% w/w] |
|---|---|
| tiotropium bromide anhydrate | 0.02 |
| HFA-134a | 99.98 |

### Formulation Example 20

| constituents | concentration [% w/w] |
|---|---|
| tiotropium bromide anhydrate | 0.02 |
| HFA-227 | 99.98 |

### Formulation Example 21

| constituents | concentration [% w/w] |
|---|---|
| tiotropium bromide anhydrate | 0.02 |
| HFA-134a | 99.98 |

### Formulation Example 22

| constituents | concentration [% w/w] |
|---|---|
| tiotropium bromide anhydrate | 0.02 |
| HFA-227 | 20.00 |
| HFA-134a | 79.98 |

### Formulation Example 23

| constituents | concentration [% w/w] |
|---|---|
| tiotropium bromide anhydrate | 0.04 |
| HFA-227 | 40.00 |
| HFA-134a | 59.96 |

### Formulation Example 24

| constituents | concentration [% w/w] |
|---|---|
| tiotropium bromide anhydrate | 0.04 |
| HFA-227 | 80.00 |
| HFA-134a | 19.96 |

It is apparent for the person of ordinary skill in the art, that the foregoing examples can be applied in analogy with one of the other crystalline forms of tiotropium bromide specified hereinbefore. In order to obtain products comprising one of the other solvates according to the invention the formulation examples 8 to 24 can easily be obtained by using one of the other crystalline solvates according to the invention instead of the tiotropium bromide anhydrate.

We claim:

1. A crystalline form of tiotropium bromide selected from the group consisting of:
crystalline tiotropium bromide anhydrate, wherein the crystalline tiotropium bromide anhydrate is characterized by a value d=5.89 Å in an X-ray powder diffraction pattern;
crystalline methanol solvate of tiotropium bromide, wherein the crystalline methanol solvate of tiotropium bromide is characterized by a value d=4.14 Å in an X-ray powder diffraction pattern;
crystalline ethanol solvate of tiotropium bromide, wherein the crystalline ethanol solvate of tiotropium bromide is characterized by a value d=4.15 Å in an X-ray powder diffraction pattern;
crystalline isopropanol solvate of tiotropium bromide, wherein the crystalline isopropanol solvate of tiotropium bromide is characterized by a value d=4.17 Å in an X-ray powder diffraction pattern;
crystalline THF solvate of tiotropium bromide, wherein the crystalline THF solvate of tiotropium bromide is characterized by a value d=4.92 Å in an X-ray powder diffraction pattern;
crystalline 1,4-dioxane solvate of tiotropium bromide, wherein the crystalline 1,4-dioxane solvate of tiotropium bromide is characterized by a value d=4.15 Å in an X-ray powder diffraction pattern;
crystalline dimethylformamide solvate of tiotropium bromide, wherein the crystalline dimethylformamide solvate of tiotropium bromide is characterized by a value d=5.69 Å in an X-ray powder diffraction pattern;
crystalline mixed methylene chloride/methyl ethyl ketone solvate of tiotropium bromide, wherein the crystalline mixed methylene chloride/methyl ethyl keton solvate of tiotropium bromide is characterized by a value d=6.56 Å in an X-ray powder diffraction pattern; and
crystalline 1-butanol solvate of tiotropium bromide, wherein the crystalline 1-butanol solvate of tiotropium bromide is characterized by a value d=4.94 Å in an X-ray powder diffraction pattern;

2. The crystalline tiotropium bromide anhydrate according to claim 1, further characterized by the values d=5.89 Å and 4.90 Å in the X-ray powder diffraction pattern.

3. The crystalline tiotropium bromide anhydrate according to claim 1, further characterized by the values d=5.89 Å, 4.90 Å and 4.84 Å in the X-ray powder diffraction pattern.

4. The crystalline methanol solvate of tiotropium bromide according to claim 1, further characterized by the values d=4.94 Å and 4.14 Å in the X-ray powder diffraction pattern.

5. The crystalline methanol solvate of tiotropium bromide according to claim 1, further characterized by the values d=4.94 Å, 4.50 Å and 4.14 Å in the X-ray powder diffraction pattern.

6. The crystalline ethanol solvate of tiotropium bromide according to claim 1, further characterized by the values d=4.46 Å and 4.15 Å in the X-ray powder diffraction pattern.

7. The crystalline ethanol solvate of tiotropium bromide according to claim 1, further characterized by the values d=4.90 Å, 4.46 Å and 4.15 Å in the X-ray powder diffraction pattern.

8. The crystalline isopropanol solvate of tiotropium bromide according to claim 1, further characterized by the values d=4.91 Å and 4.17 Å in the X-ray powder diffraction pattern.

9. The crystalline isopropanol solvate of tiotropium bromide according to claim 1, further characterized by the values d=4.91 Å, 4.48 Å and 4.17 Å in the X-ray powder diffraction pattern.

10. The crystalline THF solvate of tiotropium bromide according to claim 1, further characterized by the values d=4.92 Å and 4.15 Å in the X-ray powder diffraction pattern.

11. The crystalline THF solvate of tiotropium bromide according to claim 1, further characterized by the values d=5.80 Å, 4.92 Å and 4.15 Å in the X-ray powder diffraction pattern.

12. The crystalline 1,4-dioxane solvate of tiotropium bromide according to claim 1, further characterized by the values d=4.92 Å and 4.15 Å in the X-ray powder diffraction pattern.

13. The crystalline 1,4-dioxane solvate of tiotropium bromide according to claim 1, further characterized by the values d=5.79 Å, 4.92 Å and 4.15 Å in the X-ray powder diffraction pattern.

14. The crystalline DMF solvate of tiotropium bromide according to claim 1, further characterized by the values d=5.69 Å and 4.94 Å in the X-ray powder diffraction pattern.

15. The crystalline DMF solvate of tiotropium bromide according to claim 1, further characterized by the values d=5.69 Å, 4.94 Å and 4.11 Å in the X-ray powder diffraction pattern.

16. The crystalline mixed methylene chloride/methyl ethyl ketone solvate of tiotropium bromide according to claim 1, further characterized by the values d=6.56 Å and 4.13 Å in the X-ray powder diffraction pattern.

17. The crystalline mixed methylene chloride/methyl ethyl ketone solvate of tiotropium bromide according to claim 1, further characterized by the values d=6.56 Å, 4.22 Å and 4.13 Å in the X-ray powder diffraction pattern.

18. The crystalline 1-butanol solvate of tiotropium bromide according to claim 1, further characterized by the values d=4.94 Å and 4.17 Å in the X-ray powder diffraction pattern.

19. The crystalline 1-butanol solvate of tiotropium bromide according to claim 1, further characterized by the values d=4.94 Å, 4.51 Å and 4.17 Å in the X-ray powder diffraction pattern.

20. A method for preparing crystalline tiotropium bromide anhydrate of claim 1, comprising preparing a solution of crystalline tiotropium bromide monohydrate in dimethylformamide, adding the solution to acetonitrile to form a mixture, cooling the mixture to a temperature below 20° C. and isolating the resulting crystals.

21. A method for preparing crystalline methanol solvate of tiotropium bromide of claim 1, comprising recrystallizing an anhydrous tiotropium bromide in a methanol-containing solvent.

22. A method for preparing crystalline ethanol solvate of tiotropium bromide of claim 1, comprising recrystallizing anhydrous tiotropium bromide in an ethanol-containing solvent.

23. A method for preparing crystalline isopropanol solvate of tiotropium bromide of claim 1, comprising preparing a solution of crystalline tiotropium bromide monohydrate in isopropanol, cooling the solution to a temperature below 20° C. and isolating the resultant crystals.

24. A method for preparing crystalline THF solvate of tiotropium bromide of claim 1, comprising preparing a solution of crystalline tiotropium bromide monohydrate in a suitable alcohol, adding a solvent comprising THF to the solution and isolating the resulting crystals.

25. A method for preparing crystalline 1,4-dioxane solvate of tiotropium bromide of claim 1, comprising preparing a solution of crystalline tiotropium bromide monohydrate in a suitable alcohol, adding a solvent comprising 1,4-dioxane to the solution and isolating the resulting crystals.

26. A method for preparing crystalline DMF solvate of tiotropium bromide of claim 1, comprising preparing a solution of crystalline tiotropium bromide monohydrate in DMF, adding methyl tert-butyl ether to the solution and isolating the resulting crystals.

27. A method for preparing crystalline mixed methylene chloride/methyl ethyl ketone solvate of tiotropium bromide of claim 1, comprising preparing a solution of crystalline tiotropium bromide monohydrate in a suitable alcohol, adding a solvent comprising methylene chloride and methyl ethyl ketone to the solution to form a mixture, cooling the mixture below 20° C. and isolating the resulting crystals.

28. A method for preparing crystalline 1-butanol solvate of tiotropium bromide of claim 1, comprising preparing a solution of crystalline tiotropium bromide monohydrate in a suitable alcohol, adding a solvent comprising 1-butanol to the solution to form a mixture, cooling the mixture below 20° C. and isolating the resulting crystals.

29. The method according to claim 28, wherein the mixture is cooled below 10° C.

30. A pharmaceutical composition comprising a crystalline form of tiotropium bromide according to claim 1.

31. The pharmaceutical composition according to claim 30, further comprising one or more active ingredients selected from the group consisting of betamimetics, EGFR inhibitors, PDEIV-inhibitors, steroids, LTD4 antagonists, and mixtures thereof, optionally together with a pharmaceutically acceptable excipient.
